(19) United States
(12) Patent Application Publication
(10) Pub. No.: US 2013/0046165 A1
(43) Pub. Date: Feb. 21, 2013

(54) System for a Disposable Capacitive Bioimpedance Sensor

(71) Applicants: David E. Cassidy, Chelmsford, MA (US); Him Shing Ng, Risskov (DK)

(72) Inventors: David E. Cassidy, Chelmsford, MA (US); Him Shing Ng, Risskov (DK)

(21) Appl. No.: 13/588,675

(22) Filed: Aug. 17, 2012

Related U.S. Application Data
(60) Provisional application No. 61/524,468, filed on Aug. 17, 2011.

(51) Int. Cl. A61B 5/053

(52) U.S. Cl. 600/386; 600/547

(57) ABSTRACT

A system for a disposable capacitive bioimpedance sensor is used to collect bioimpedance data from an electric current traveling into or through an organism's tissue, where the bioimpedance data can be used to make medical analysis. The system mainly comprises at least one disposable sensor, at least one communication channel, a sensing circuit, and an instrument portion. The disposable sensor and the sensing circuit are used to retrieve the bioimpedance data from the organism's tissue, which is then sent to the instrument portion through the communication channel in order to be analyzed and processed. The disposable sensor includes a plurality of electrodes, an electrode support layer, and a capacitive signal receptor. The electrodes deliver the electrical current to the organism's tissue, and the capacitive signal receptor allows the electrical current to be properly sent or received through the organism's tissue. The electrode support layer holds the electrodes in place.

The current application claims a priority to the U.S. Provisional Patent application Ser. No. 61/524,468 filed on Aug. 17, 2011.

### FIELD OF THE INVENTION

The present invention relates generally to a method and apparatus for acquiring physiological and biological data. More specifically, the present invention is a method and apparatus to detect physiological and biological signal from a patient using a two-part system of a disposable sensor unit and a signal pre-processing unit.

### BACKGROUND OF THE INVENTION

Physiological and biological information extracted from organisms can provide valuable insights for facilitating interventional strategies in cases of pathological conditions. An interface in form of a sensor is often used to acquire biomedical data. The acquired biomedical data is often fed into a system to be processed into useful information. An organism is mainly composed of cells, tissue, and body fluid, which are capable of transporting and/or storing electrons. The compositions of different cells, tissues, or body parts have their own unique electrical impedance characteristics. Bioimpedance analysis is a method of extracting the impedance characteristics from a living organism by using the living organism as an electrical conductor, which can be used to deduce physiological and biological information. Bioimpedance analysis is a well established methodology that requires an interface, i.e. a sensor, to acquire data from the living organism.

The present invention is a system using disposable capacitive impedance sensor to acquire physiological and biological data for various applications. The present invention also includes a special signal pre-processor, which is operatively coupled to the novel sensor.

### BRIEF DESCRIPTION OF THE DRAWINGS

FIG. 1 depicts how multiple electrodes can be arranged in a 1-dimensional array to form a sensor.

FIG. 2 depicts how multiple electrodes can be arranged in a 2-dimensional array to form a sensor.

FIG. 3 depicts how multiple electrodes can be arranged in a 3-dimensional array to form a sensor.

FIG. 4 is a simple schematic of the present invention and the interface to a human body.

FIG. 5 is a cross-sectional view of the first configuration of the sensor.

FIG. 6 is a cross-sectional view illustrating how the electrodes on the first configuration of the sensor are aligned to the subject of interest and how the electric current from the electrodes travels around an organism's tissue.

FIG. 7A is a cross-sectional view of the second configuration of the sensor.

FIG. 7B is a cross-sectional view illustrating how the electrodes on the second configuration of the sensor are aligned to the subject of interest and how the electric current from the electrodes travels through an organism's tissue.

FIG. 8 is a cross-sectional view of the first configuration of the sensor with each electrode having a sensing circuit.

FIG. 9A is a schematic of the first embodiment of the sensing circuit.

FIG. 9B is a schematic of an alternate embodiment of the sensing circuit, which relates to the first embodiment of the sensing circuit.

FIG. 10 is a schematic of the second embodiment of the sensing circuit.

FIG. 11 is a schematic of the third embodiment of the sensing circuit.

FIG. 12 is a schematic depicting an alternating current stimulus with multiple frequencies.

FIG. 13 is a circuit diagram of one possible signal processing method.

FIG. 14 is a circuit diagram of another possible signal processing method.

### DETAILED DESCRIPTIONS OF THE INVENTION

All illustrations of the drawings are for the purpose of describing selected versions of the present invention and are not intended to limit the scope of the present invention.

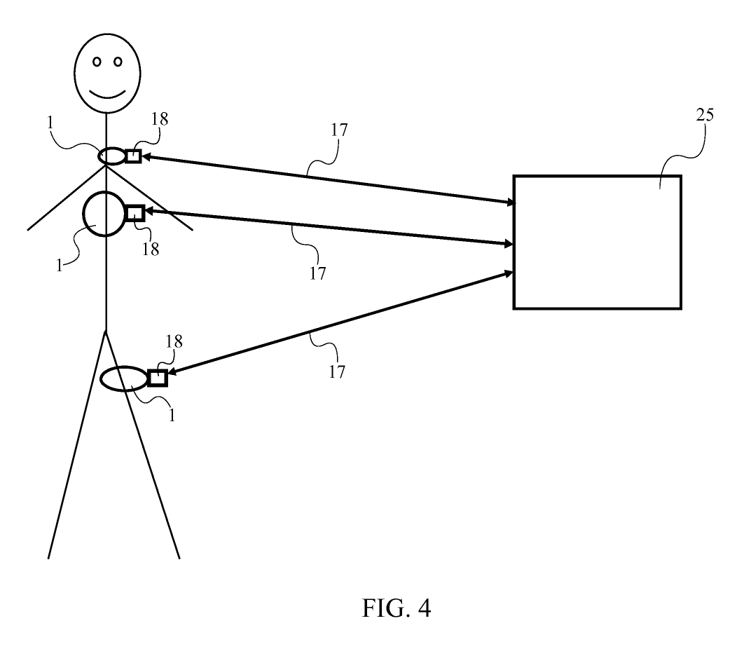

As can be seen in FIG. 4, the present invention is a system for a disposable capacitive bioimpedance sensor and is used to perform bioimpedance analysis on an organism's tissue. The present invention is used, but not limited to, the following applications: local arterial stiffness assessment, pulse wave velocity measurement, blood pressure measurement, heart rate measurement, tumor detection, shrapnel or bullet fragment detection, and deep vein thrombosis diagnosis. The present invention comprises an at least one disposable sensor 1, a communication connection 17, a sensing circuit 18, and an instrument portion 25. The at least one disposable sensor 1 is used to retrieve the bioimpedance data from the organism's tissue. The sensing circuit 18 provides and manages the alternating current (AC) electrical stimulus passing through the at least one disposable sensor 1. In addition, the sensing circuit 18 creates a signal containing the bioimpedance data. The instrument portion 25 allows the present invention to process the signal into user readable data, which can be used for medical measurements or diagnosis. The communication connection 17 is used to communicably couple the at least one disposable sensor 1 through the sensing circuit 18 to the instrument portion 25.

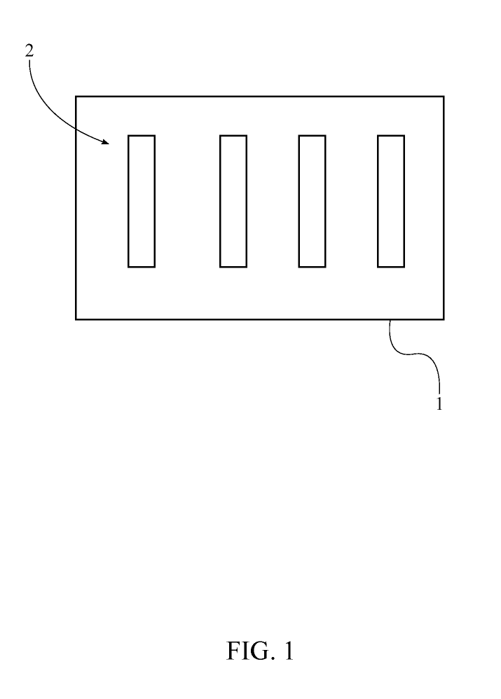

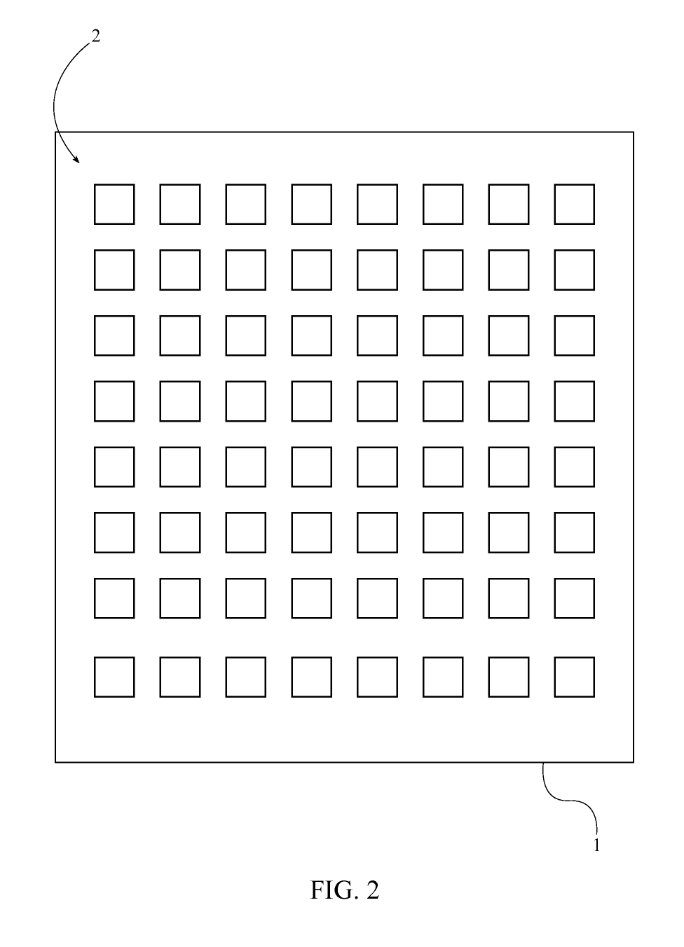

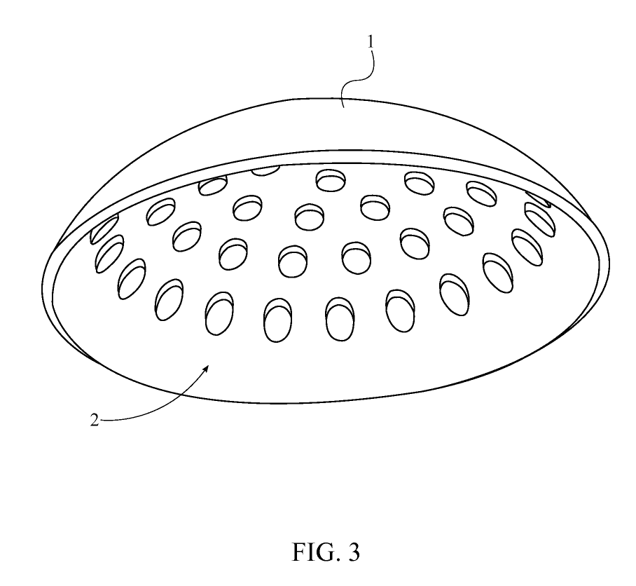

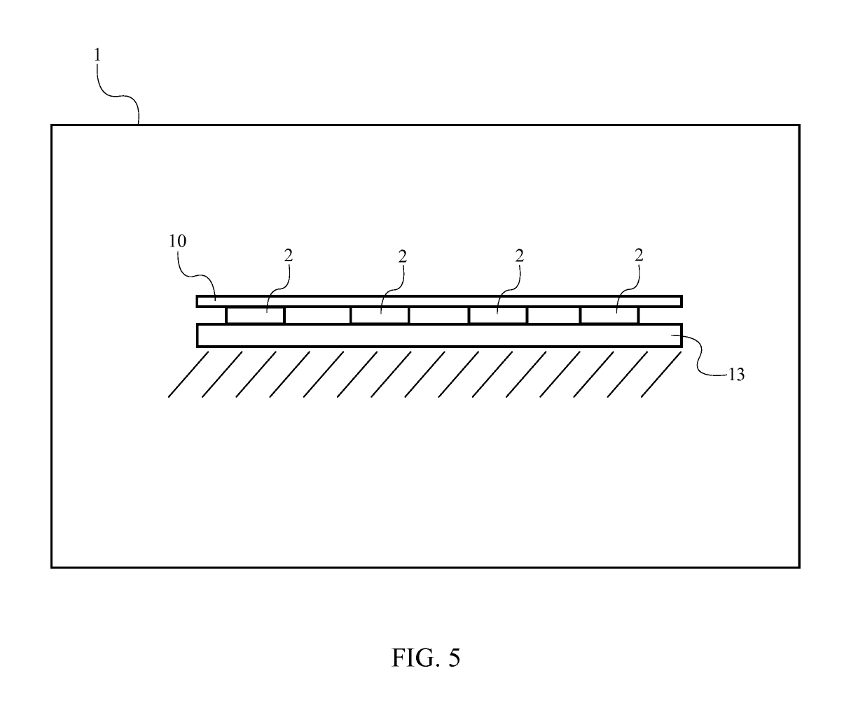

The at least one disposable sensor 1 capacitively couples to the organism's tissue in order to retrieve the bioimpedance data. The at least one disposable sensor 1 is placed over the measuring site of the organism's tissue similar to electrocardiography (ECG) sensor. Unlike the ECG sensor, the at least one disposable sensor 1 of the present invention contains multiple electrodes and is non-conductive. In the first configuration shown in FIG. 5, the at least one disposable sensor 1 comprises a plurality of electrodes 2, an electrode support layer 10, and a capacitive signal receptor 13. The plurality of electrodes 2 allows the at least one disposable sensor 1 to send and receive an electric current through the organism's tissue. The plurality of electrodes 2 can be positioned as either a one-dimensional array shown in FIG. 1, a two-dimensional array shown in FIG. 2, or a three-dimensional array shown in FIG. 3, which are sized and shaped in such a way to maximize signal sensitivity. In the preferred embodiment of the present invention, the plurality of electrodes 2 can constructed of, but not limited to, metal or carbon. The plurality of electrodes 2 is connected onto the electrode support layer 10, which is used to properly arrange and maintain the geometric configuration of the plurality of electrodes 2. In the preferred embodiment, the electrode support layer 10 is flexible and non-conductive and can be made of a material with a low dielectric constant such as, but is not limited to, plastic films composed of polyester or polycarbonate. The capacitive signal receptor 13 is connected across the plurality of electrodes 2, which positions the plurality of electrodes 2 in between the electrode support layer 10 and the capacitive signal receptor 13. The capacitive signal receptor 13 makes contact with the organism's tissue and allows the at least one disposable sensor 1 to transfer the electrical current between the plurality of electrodes 2 and the organism's tissue. The capacitive signal receptor 13 has a high dielectric constant in order to enhance the ability of the capacitive signal receptor 13 to transfer the electrical current between the plurality of electrodes 2 and the organism's tissue. In the preferred embodiment, the capacitive signal receptor 13 is a thin, flexible, and non-electrically conductive material such as, but not limited to, hydrogel, hypoallergenic adhesives or tapes, and acrylic adhesives or tapes. Also in the preferred embodiment, the capacitive signal receptor 13 is made of a material with temporary adhesive qualities so that the at least one disposable sensor 1 can stick to the surface of the organism's tissue.

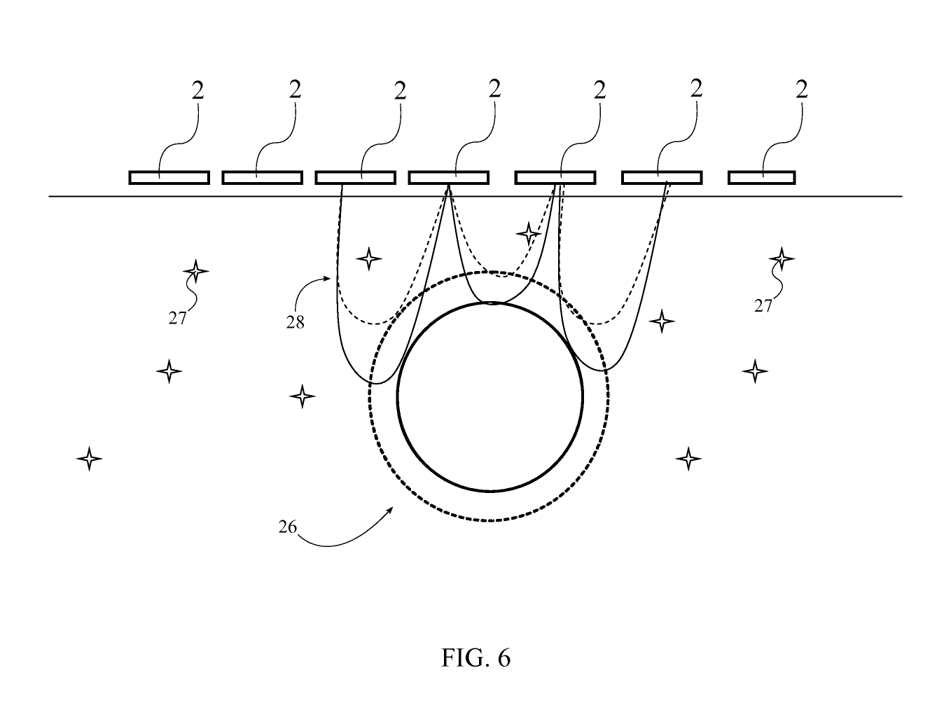

For the first configuration of the at least one disposable sensor 1, FIG. 6 shows a cross-sectional view of how a one-dimensional array of electrodes is aligned in a row perpendicular to the subject of interest 26 embedded within the organism's tissue 27. However, the arrangement of the plurality of electrodes 2 is not limited to the row configuration of the one-dimensional array. When the subject of interest 26 inside the organism's tissue 27 is its original form, the longer electrical current flux lines 28 are propagating through the organism's tissue 27. Once the subject of interest 27 inside the organism's tissue 27 expands from its original form, the increase in volume for the subject of interest 26 will alter the propagation of the electrical current flux lines 28 through the organism's tissue. As a result, the capacitance changes between each of the plurality of electrodes 2. Different kinds of tissue, such as blood and skin, respond differently at different frequencies, and, therefore, it is desirable to apply more than one frequency to differentiate these different kinds of tissue.

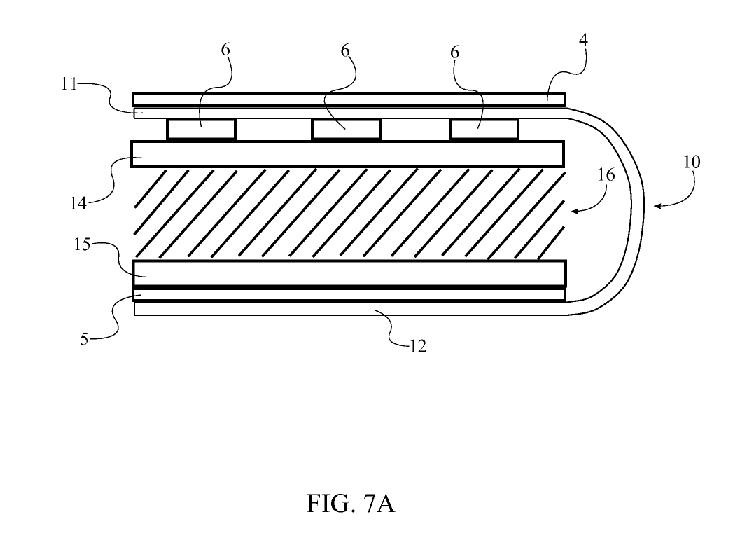

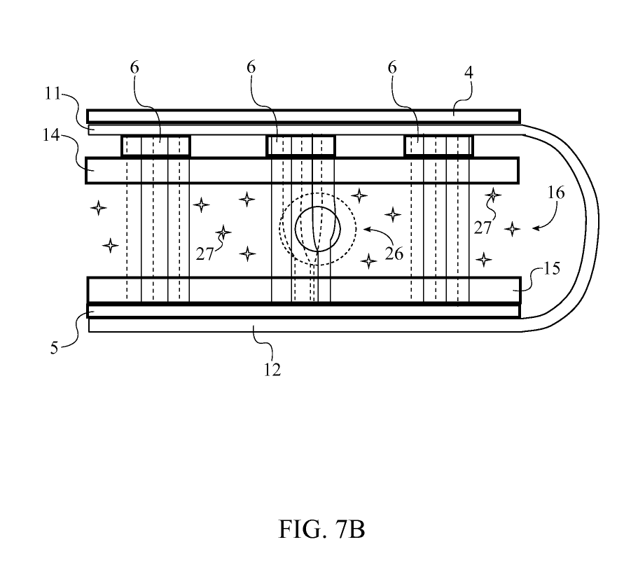

As can be seen in FIG. 7A, the second configuration of the at least one disposable sensor 1 is used to clasp the organism's tissue from two sides as opposed to the first configuration, which only touches the organism's tissue from one side. In the second configuration, the at least one disposable sensor 1 comprises a plurality of electrodes 2, an electrode support layer 10, a first capacitive signal receptor 14, and a second capacitive signal receptor 15. For the second configuration, the electrode support layer 10 comprises a first brace portion 11 and a second brace portion 12, which are used to properly support the at least one disposable sensor 1 around the tissue insertion area 16. The organism's tissue is placed within the tissue insertion area 16 to ensure the accurate measurements are taken. The first brace portion 11 is located adjacent to the tissue insertion area 16, which allows the first brace portion 11 to support the organism's tissue from one side. The second brace portion 12 is located adjacent to the tissue insertion area 16 opposite to the first brace portion 11, which allows the second brace portion 12 support the organism's tissue from the other side. For both the first configuration and the second configuration, the plurality of electrodes 2 comprises a set of stimulating electrodes 3 and a set of sensing electrodes 6, which are separated by how the set of stimulating electrodes 3 is connected to the sensing circuit 18 and how the set of sensing electrodes 6 is connected to the sensing circuit 18.

For the second configuration, the set of stimulating electrodes 3 comprises a first stimulating electrode 4 and a second stimulating electrode 5, which are meant to be positioned on the at least one disposable sensor 1 in a certain manner. Thus, the set of sensing electrodes 6 is able to be positioned in between the first stimulating electrode 4 and the second stimulating electrode 5, which allows the second configuration of the at least one disposable sensor 1 to pass the electric current from one side of the organism's tissue to the other side of the organism's tissue. More specifically, the first stimulating electrode 4 is connected onto to the first brace portion 11 opposite to the tissue insertion area 16, and the set of sensing electrodes 6 is connected on the first brace portion 11 opposite to the first stimulating electrode 4, which allows both the first stimulating electrode 4 and the set of sensing electrodes 6 to be supported by the first brace portion 11. The capacitance between the set of sensing electrodes 6 and the first stimulating electrode 4 remains constant because of the uniformed spacing caused by the thickness of the first brace portion 11. The first capacitive signal receptor 14 is connected across the set of sensing electrodes 6, which positions the set of sensing electrodes 6 in between the first brace portion 11 and the first capacitive signal receptor 14 and allows both the first stimulating electrode 4 and the set of sensing electrodes 6 to adequately send or receive the electric current through the organism's tissue. The second capacitive signal receptor 15 is positioned adjacent to the tissue insertion area 16 opposite to the first capacitive signal receptor 14 so that the second capacitive signal receptor 15 also sends or receives the electric current through the organism's tissue. The second stimulating electrode 5 is connected in between to both the second capacitive signal receptor 15 and the second brace portion 12, which allows the second stimulating electrode 5 to be supported by the second brace portion 12 and to be electrically coupled to the organism's tissue by the second capacitive signal receptor 15. Similar to FIG. 6, the expansion of the subject of interest 26 within the organism's tissue 27 is shown in FIG. 7B to alter the electric current flux lines 28 that are travelling from the first stimulating electrode 4 to the second stimulating electrode 5.

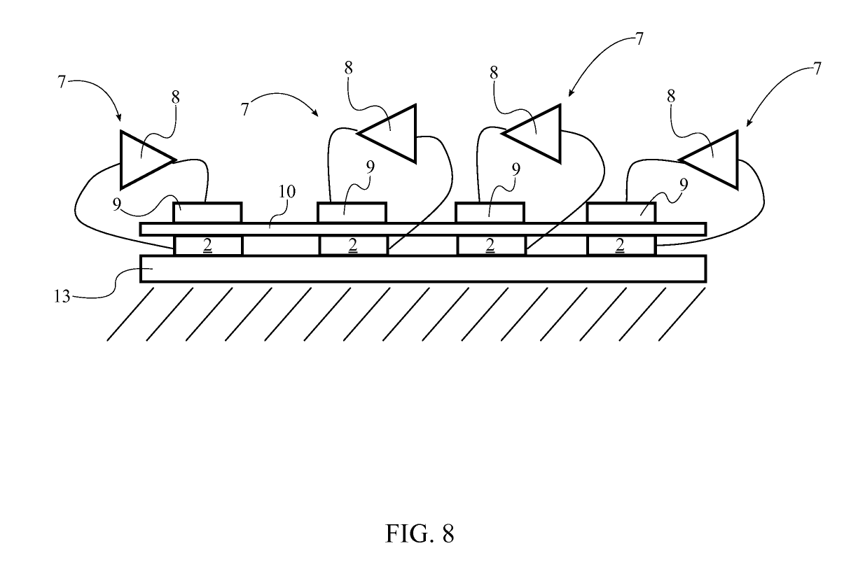

For the second configuration, the first stimulating electrode 4 and the second stimulating electrode 5 also act as an electrostatic shield to minimize measurement noise from the set of sensing electrodes 6. However, the first configuration does not use the same arrangement as the second configuration and may require a plurality of shielding circuits 7 to minimize measurement noise to the plurality of electrodes 2. Whether or not each electrode 2 requires a shielding circuit 7 depends on the sensitivity of the plurality of electrodes 2 and the noise from the surrounding environment. As can be seen in FIG. 8, each shielding circuit 7 for each electrode comprises a buffer 8 and a conductor 9. The conductor 9 is connected to the electrode support layer 10 opposite to their corresponding electrode 2. Each conductor 9 should be relatively similar in size to their corresponding electrode 2. If a conductor 9 is relatively larger than its corresponding electrode 2, then the electric current could be diverted from the electrode 2 to the conductor 9. If a conductor 9 is relatively smaller than its corresponding electrode 2, then the shielding circuit 7 will not be as effective for that electrode 2. In addition, each conductor 9 is electronically connected to their corresponding electrode 2 through the buffer 8. Each conductor 9 would be driven by the physiological signal travelling from its corresponding electrode 2 and through its buffer 8.

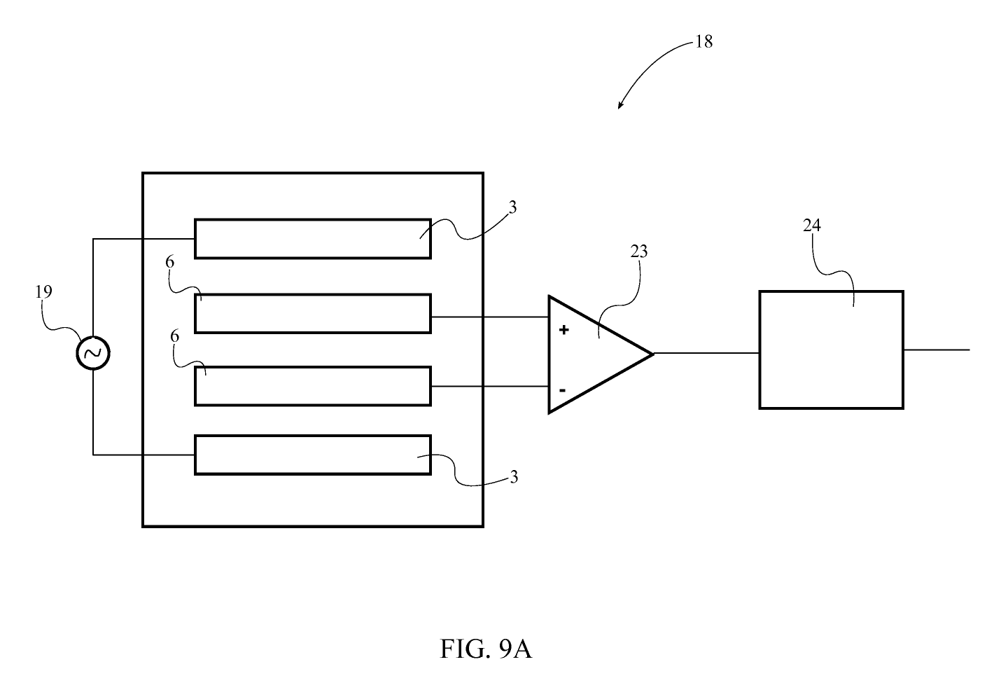

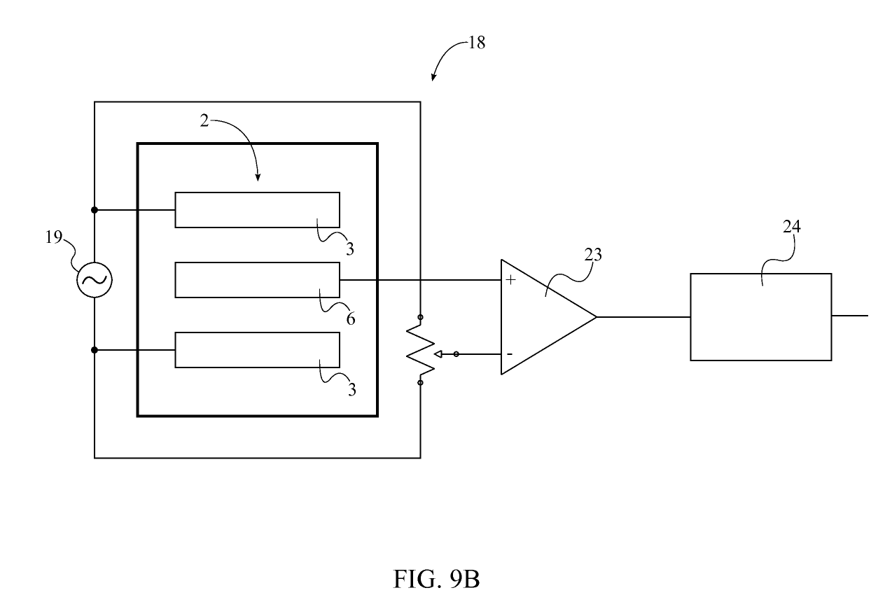

As can be seen in FIG. 9A, 10, and 11, the sensing circuit 18 can be configured with a number of different components and arrangements, but every configuration for a sensing circuit 18 comprises an AC stimulus 19, an at least one amplifier 23, and an at least one demodulating processor 24. The AC stimulus 19 is electrically connected to the set of stimulating electrodes 3 so that the AC stimulus 19 can supply the electrical current to the set of stimulating electrodes 3. The AC stimulus 19 allows the set of stimulating electrodes 3 to send a regulated electrical current through the organism's tissue, which is independent from the electrical impedance of the set of stimulating electrodes 3. The AC stimulus 19 uses at least one frequency with amplitude(s) that are safe for contact with the organism's tissue. The typical frequencies used for the AC stimulus 19 are between 1 kilohertz (kHz) and 10 megahertz (MHz). The at least one amplifier 23 is electrically connected to the set of sensing electrodes 6. The voltage across the organism's tissue is recorded by the at least one amplifier 23, which is relatively unaffected by the impedance of the set of stimulating electrodes 3. The at least one demodulating processor 24 electronically connected to the at least one amplifier 23. Once the voltage reading is received by the set of sensing electrodes 6 and is amplified by the at least one amplifier 23, the at least one demodulating processor 24 will extract the physiological signal from the voltage reading. The at least one demodulating processor 24 is capable of decreasing the overall noise from the system because the signal from the AC stimulus 19 is known. The physiological signal outputted by the at least one demodulating processor 24 is interpreted as the impedance of the organism's tissue.

The first embodiment of the sensing circuit 18 has the simplest configuration and is shown in FIG. 9A. For the first embodiment, the set of stimulating electrodes 3 requires at least one pair of electrodes, and the set of sensing electrodes 6 also requires at least one pair of electrodes. The first embodiment is used to measure the absolute impedance through the organism's tissue so that the set of stimulating electrodes 3 have fixed spacing in between them and must be precisely positioned over the object being measured within the organism's tissue. An alternation of the first embodiment of the sensing circuit 18 is shown in FIG. 9B, one of the sensing electrodes 6 is removed so that the negative reference voltage is measured directly from the AC stimulus 19.

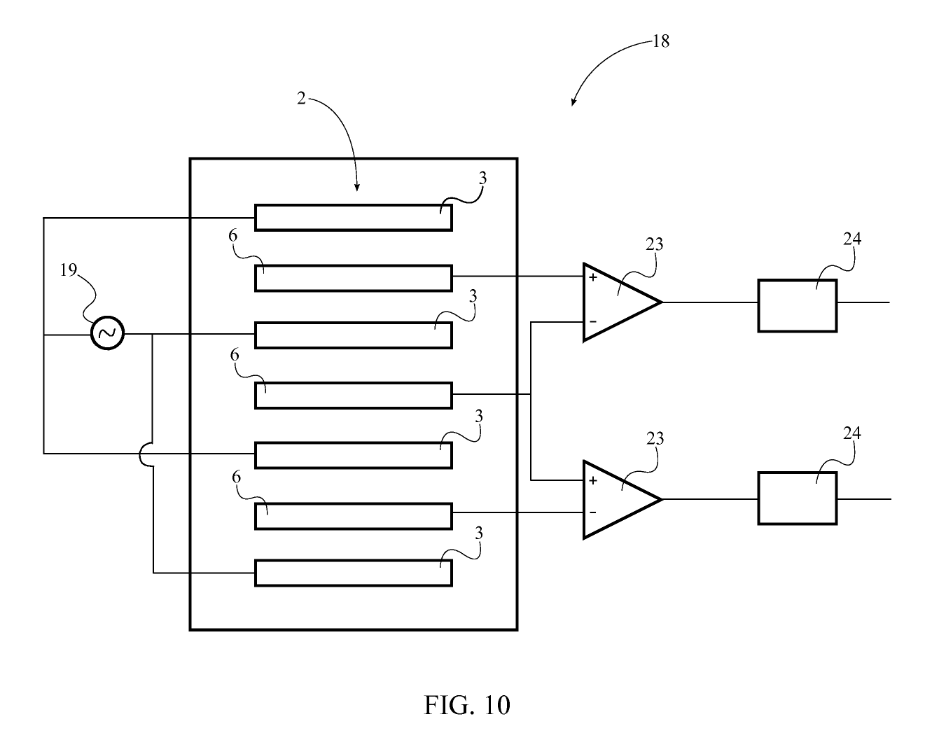

The second embodiment of the sensing circuit 18 measures relative impedance by using at least one or more Wheatstone bridges, which are shown in FIG. 10. Each Wheatstone bridge includes at least a pair of stimulating electrodes 3, a pair of sensing electrodes 6, one amplifier 23, and one demodulating processor 24. The second embodiment allows the sensing circuit 18 to make simultaneous measurements at different locations on the organism's tissue by using multiple Wheatstone bridges so that the positioning of each pair of sensing electrodes 6 is not critical to measuring a subject of interest within the organism's tissue because the multiple Wheatstone bridges allow the at least one disposable sensor 1 to cover a much larger area.

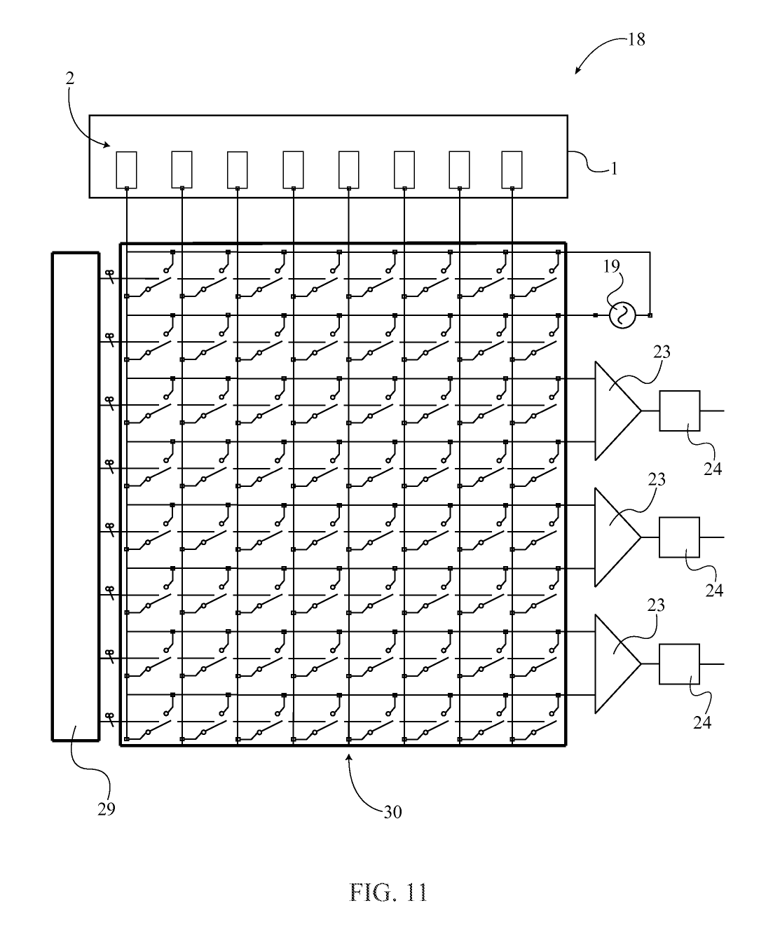

The third embodiment of the sensing circuit 18 allows the plurality of electrodes 2 to alternate between a stimulating electrode and a sensing electrode by means of a multiplexing device 30 such as, but not limited to, a cross point switch, which is shown in FIG. 11. A multiplexing device 30 allows any of the plurality of electrodes 2 to alternate between being either a stimulating electrode or a sensing electrode. The multiplexing device 30 allows the AC stimulus 19 to electrically connect to each of the plurality of electrodes 2. In addition, the multiplexing device allows the at least one amplifier 23 to electrically connect to each of the plurality of electrodes 2. For the third embodiment, any of the plurality of electrodes 2 can be assigned to be sensing electrodes in order to increase or decrease the surface area of the organism's tissue that is being measured. A larger width between two sensing electrodes allows the at least one disposable sensor 1 to take deeper measurements within the organism's tissue. A smaller width between two sensing electrodes allows the at least one disposable sensor 1 to take more detailed measurements within the organism's tissue. The selection, the spacing, and the position of plurality of electrodes 2 can be dynamically modified by the controller 29 of the multiplexing device 30, which makes the position of each individual electrode on the organism's tissue less important. The controller 29 is also electronically connected to each of the plurality of electrodes 2 through the multiplexing device 30.

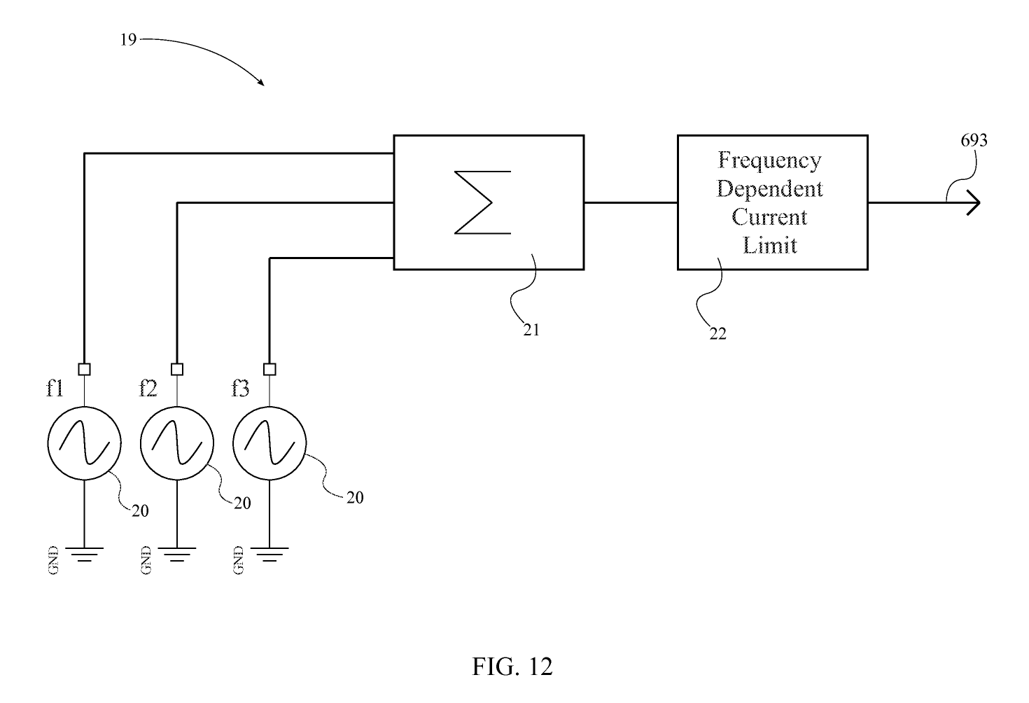

As can be seen in FIG. 12, the electric current provided by the AC stimulus 19 can be a summation of multiple signal sources, where each signal source has a differing carrier frequency. Consequently, the AC stimulus 19 comprises a plurality of voltage sources 20, a signal summation block 21, and the current limitation block 22. The plurality of voltages sources 20 is used to produce each of the multiple signals at their differing carrier frequencies. Each of the plurality of voltage sources 20 is electrically connected to the signal summation block 21 so that the signal summation block 21 can combine the multiple signals into one input signal for the set of stimulating electrodes 3. The signal summation block 21 is also electrically connected to the current limitation block 22, which limits the electrical current of the one input signal to safe and acceptable levels for each frequency.

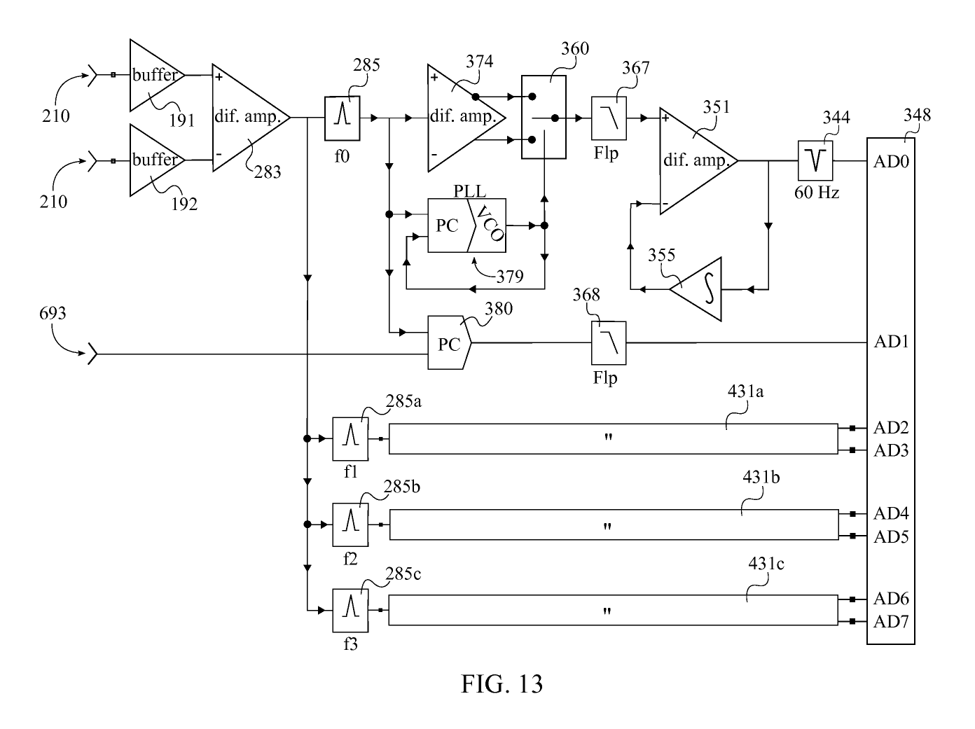

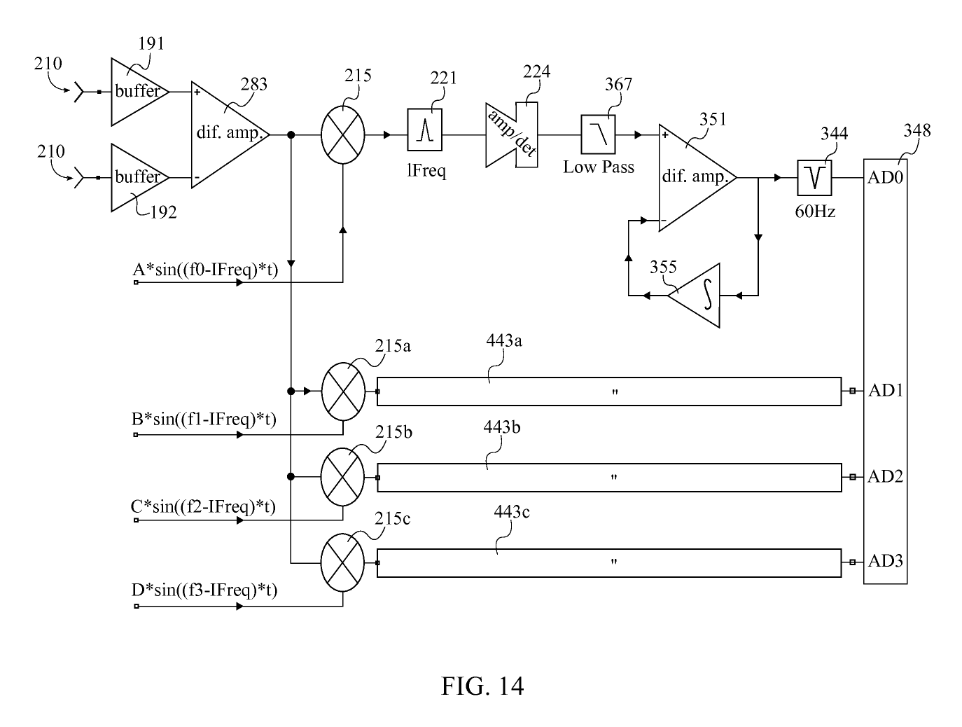

FIG. 13 and FIG. 14 depict two possible signal processing methods. It is understood by those knowledgeable in the art that there are a variety of solutions. These methods can be accomplished by discrete electronic components or by software in a computer.

For FIG. 13, a first buffer 191 and a second buffer 192 amplify the current of electrode signals 210 with a voltage gain of 1. A first differential amplifier 283 amplifies the difference of the first buffer 191 and the second buffer 192. The band pass filter 285 is set to filter one of the frequencies from the plurality of voltage sources 20. The frequency of the band pass filter 285 can be set dynamically by a controller or fixed by electronic component values. The output of the band pass filter 285 is used as a signal input to a phase lock loop 379 and is also amplified by a second differential amplifier 374. The second differential amplifier 374 has two outputs, which are 180 degrees out of phase. The phase lock loop 379 provides a stable duty cycle clock signal at the same frequency and phase as the desired AC carrier signal frequency 693 from the plurality of voltage sources 20, which can also be seen in FIG. 12, or the switch 360 independent of noise. The switch 360 acts as a synchronous rectifier, rectifying only the desired AC carrier signal frequency 693. This process is sometimes referred to in the trade as a lock amplifier. The output signal of the switch 360 drives a first low pass filter 367 set to filter the physiological/biological signals to be measured. The third differential amplifier 351 multiplies the output of the first low pass filter 367 to a measurable level by the analog-to-digital (A/D) converter 348. The output of the third differential amplifier 351 is also fed into an integrator 355 and fed back into the negative input of the third differential amplifier 351 to eliminate the direct current (DC) offset found in the rectified signal. The frequency cutoff of the integrator 355 is set to the lowest frequency of the physiological signal to be measured. The output of the third differential amplifier 351 is filtered for line frequency noise by a notch filter 344. The possible line frequencies are, but not limited to, 50 or 60 Hz. The line filtered signal is then fed into an A/D converter 348 to be processed by a computer. A secondary path, providing measured signal phase, comprises a phase comparator 380 and a second low pass filter 368. One input comes from the AC carrier signal frequency 693 while a second input comes from the band pass filter 285. The low pass filter 368 converts the digital output of the phase comparator 380 to an analog signal for A/D converter 348. Each of the plurality of parallel paths comprises a path band pass filter 285a, 285b, 285c and a path processor 431a, 431b, 431c, which extract the desired physiological signal from the different frequencies of the AC stimuli 20.

FIG. 14 depicts a second method of signal processing. For the second method, the first buffer 191 and the second buffer 192 amplify the current of electrode signals 210 with a voltage gain of 1. The initial differential amplifier 283 amplifies the difference of the first buffer 191 and the second buffer 192. The amplified signal is fed into a multiplier 215, where the amplified signal is multiplied by a reference signal in the form A*sin ((f₀−IFreq)*t) volts, where: 1.) A is the amplitude of the signal; 2.) f₀ is the AC carrier signal frequency 693; 3.) IFreq is the intermediate frequency to down convert f₀; and 4.) t is time. The fundamental output of the multiplier 215 is a signal that is composed of f₀, IFreq, and the physiological signal. Frequency component, f₀, of the fundamental output is filtered out by a band pass filter 221 which is set to IFreq. The output of the band pass filter 221 is then fed into an amplifier-rectifier block 224. The rectified signal is filtered through the low pass filter 367 eliminating frequencies higher than the physiological signal to be measured. The subsequent amplifier 351 multiplies the output of the low pass filter 367 to a measurable level of the A/D converter 348. The output of the subsequent amplifier 351 is also fed into the integrator 355 and then fed back into the negative input of the subsequent amplifier 351 to eliminate the DC offset caused by the rectification process. The frequency cutoff of the integrator 355 is set to the lowest frequency of the physiological signal to be measured. The output of the subsequent amplifier 351 is filtered for line frequency noise by the notch filter 344. The possible line frequencies are, but not limited to, 50 or 60 Hz. The line filtered signal is then fed into an A/D converter 348 to be processed by a computer. Each of the plurality of parallel paths comprises a path multiplier 215a, 215b, 215c and a path processor 443a, 443b, 443c, which extract the desired physiological signal from the different frequencies of the AC stimuli 20.

Although the invention has been explained in relation to its preferred embodiment, it is to be understood that many other possible modifications and variations can be made without departing from the spirit and scope of the invention as hereinafter claimed.

What is claimed is:

1. A system for a disposable capacitive bioimpedance sensor comprises,
an at least one disposable sensor;
a communication connection;
a sensing circuit;
an instrument portion;
said at least one disposable sensor comprises a plurality of electrodes, an electrode support layer, and a capacitive signal receptor;
said sensing circuit comprises an AC stimulus, an at least one amplifier, and an at least one demodulating processor;
said AC stimulus comprises a plurality of voltage sources, a signal summation block, and a current limitation block; and
said instrument portion being communicably coupled to said at least one disposable sensor through said sensing circuit by said communication connection.

2. The system for a disposable capacitive bioimpedance sensor as claimed in claim 1 comprises,
said plurality of electrodes being connected onto said electrode support layer;
said capacitive signal receptor being connected across said plurality of electrodes; and
said plurality of electrodes being positioned in between said electrode support layer and said capacitive signal receptor.

3. The system for a disposable capacitive bioimpedance sensor as claimed in claim 2, wherein said plurality of electrodes being positioned as a one-dimensional array.

4. The system for a disposable capacitive bioimpedance sensor as claimed in claim 2, wherein said plurality of electrodes being positioned as a two-dimensional array.

5. The system for a disposable capacitive bioimpedance sensor as claimed in claim 2, wherein said plurality of electrodes being positioned as a three-dimensional array.

6. The system for a disposable capacitive bioimpedance sensor as claimed in claim 2 comprises,
a plurality of shielding circuits;
each of said plurality of shielding circuits comprises a conductor, a buffer, and a corresponding electrode from said plurality of electrodes;
said conductor being electronically connected to said corresponding electrode through said buffer; and
said conductor being connected to said electrode support layer opposite to said corresponding electrode.

7. The system for a disposable capacitive bioimpedance sensor as claimed in claim 1 comprises,
said plurality of electrodes comprises a set of stimulating electrodes and a set of sensing electrodes;
said AC stimulus being electrically connected to said set of stimulating electrodes;
said at least one amplifier being electrically connected to said set of sensing electrodes; and
said at least one demodulating processor being electronically connected to said at least one amplifier.

8. The system for a disposable capacitive bioimpedance sensor as claimed in claim 1 comprises,
said sensing circuit further comprises a multiplexing device and a controller;
said AC stimulus being electrically connected to each of said plurality of electrodes through said multiplexing device;
said controller being electronically connected to each of said plurality of electrodes through said multiplexing device;
said at least one amplifier being electrically connected to each of said plurality of electrodes through said multiplexing device; and
said at least one demodulating processor being electronically connected to said at least one amplifier.

9. The system for a disposable capacitive bioimpedance sensor as claimed in claim 8, wherein said multiplexing device is used to alternate each of said plurality of electrodes between being a stimulating electrode and being a sensing electrode.

10. The system for a disposable capacitive bioimpedance sensor as claimed in claim 1 comprises,
said AC stimulus comprises a plurality of voltage sources, a signal summation block, and a current limitation block;
each of the plurality of voltage sources being electrically connected to said signal summation block; and
said signal summation block being electrically connected to said current limitation block.

11. A system for a disposable capacitive bioimpedance sensor comprises,
an at least one disposable sensor;
a communication connection;
a sensing circuit;
an instrument portion;
said at least one disposable sensor comprises a plurality of electrodes, an electrode support layer, a tissue insertion area, a first capacitive signal receptor, and a second capacitive signal receptor;
said sensing circuit comprises an AC stimulus, an at least one amplifier, and an at least one demodulating processor;
said electrode support layer comprises a first brace portion and a second brace portion;
said AC stimulus comprises a plurality of voltage sources, a signal summation block, and a current limitation block; and
said instrument portion being communicably coupled to said at least one disposable sensor through said sensing circuit by said communication connection.

12. The system for a disposable capacitive bioimpedance sensor as claimed in claim 11 comprises,
said set of stimulating electrodes comprises a first stimulating electrode and a second stimulating electrode;
said first brace portion being located adjacent to said tissue insertion area;
said second brace portion being located adjacent to said tissue insertion area opposite to said first brace portion; and
said set of sensing electrodes being positioned in between said first stimulating electrode and said second stimulating electrode.

13. The system for a disposable capacitive bioimpedance sensor as claimed in claim 12 comprises,
said first stimulating electrode being connected onto to said first brace portion opposite said tissue insertion area;
said set of sensing electrodes being connected onto said first brace portion opposite to said first stimulating electrode;
said first capacitive signal receptor being connected across said set of sensing electrodes;
said set of sensing electrodes being positioned in between said first brace portion and said first capacitive signal receptor;
said second capacitive signal receptor being positioned adjacent to tissue insertion area opposite to said first capacitive signal receptor; and
said second stimulating electrode being connected in between to both said second capacitive signal receptor and said second brace portion.

14. The system for a disposable capacitive bioimpedance sensor as claimed in claim 12, wherein said plurality of electrodes being positioned as a one-dimensional array.

15. The system for a disposable capacitive bioimpedance sensor as claimed in claim 12, wherein said plurality of electrodes being positioned as a two-dimensional array.

16. The system for a disposable capacitive bioimpedance sensor as claimed in claim 12, wherein said plurality of electrodes being positioned as a three-dimensional array.

17. The system for a disposable capacitive bioimpedance sensor as claimed in claim 11 comprises,
said plurality of electrodes comprises a set of stimulating electrodes and a set of sensing electrodes;
said AC stimulus being electrically connected to said set of stimulating electrodes;
said at least one amplifier being electrically connected to said set of sensing electrodes; and
said at least one demodulating processor being electronically connected to said at least one amplifier.

18. The system for a disposable capacitive bioimpedance sensor as claimed in claim 11 comprises,
said sensing circuit further comprises a multiplexing device and a controller;
said AC stimulus being electrically connected to each of said plurality of electrodes through said multiplexing device;
said controller being electronically connected to each of said plurality of electrodes through said multiplexing device;
said at least one amplifier being electrically connected to each of said plurality of electrodes through said multiplexing device; and
said at least one demodulating processor being electronically connected to said at least one amplifier.

19. The system for a disposable capacitive bioimpedance sensor as claimed in claim 18, wherein said multiplexing device is used to alternate each of said plurality of electrodes between being a stimulating electrode and being a sensing electrode.

20. The system for a disposable capacitive bioimpedance sensor as claimed in claim 11 comprises,
said AC stimulus comprises a plurality of voltage sources, a signal summation block, and a current limitation block;
each of the plurality of voltage sources being electrically connected to said signal summation block; and
said signal summation block being electrically connected to said current limitation block.
